(19) United States
(12) Patent Application Publication
(10) Pub. No.: US 2012/0052525 A1
(43) Pub. Date: Mar. 1, 2012

(54) POLYMERS FOR GROWING CELLS

(76) Inventors: David C. Hay, Edinburgh (GB); John P. Iredale, Edinburgh (GB); Mark Bradley, Edinburgh (GB); Juan J. Diaz-Mochon, Edinburgh (GB); Salvatore Pernagallo, Edinburgh (GB)

(21) Appl. No.: 13/257,747

(22) PCT Filed: Mar. 19, 2010
(86) PCT No.: PCT/GB2010/000523

§ 371 Date: Nov. 18, 2011

(30) Foreign Application Priority Data
Mar. 20, 2009 (GB) 09048374.9

(51) Int. Cl. C12Q 1/02; C12M 3/00; C08G 18/76; C12N 11/08; C12N 5/071

(52) U.S. Cl. 435/29; 435/180; 435/396; 528/76; 528/70; 435/289.1

(57) ABSTRACT

The present invention provides a polymer substrate for use in the attachment and functioning of hepatocyte and hepatocyte like cells. In particular, the polymer substrate is a polyurethane polymer.

### FIELD OF THE INVENTION

The present invention relates to the provision of certain polymers to which hepatocytes are able to attach and display hepatocyte function for a period of time. There is also provided use of certain polymers for attachment and maintenance of function of hepatocytes. There is further provided apparatus formed of, or comprising a coating of the polymers of the present invention for use in the attachment and maintenance of functional hepatocytes.

### BACKGROUND OF THE INVENTION

The cost of drug development is heavily influenced by compound attrition rate. For every new drug that reaches the market, ˜5000 to 10,000 compounds have been tested in preclinical trials with ˜250 reaching animal trials. Following animal trials ˜5 possible drugs make it to full-scale human clinical trials with only 1 obtaining final approval [1]. These figures demonstrate there is a clear requirement for developing more accurate predictive toxicity models. The generation of human hepatocyte like cells (HLCs) from human embryonic stem cells (hESCs) and human induced pluripotent stem cells (hiPSCs) is one such approach. We have recently developed in vitro models of human liver function [2-6], although efficient maintenance of their long-term hepatocyte function has proven elusive.

### SUMMARY OF THE INVENTION

The present invention is based on the identification of a class of polymers which have been shown to possess the features of allowing attachment of hepatocyte cells thereto, which attached cells display good hepatocyte functional properties.

In a first aspect there is provided a polymer substrate for use in the attachment and functioning of hepatocyte and hepatocyte like cells.

The present inventors have observed that the polyurethane surface, formed by polymerising PHNGAD, MDI and an extender, provides a supportive effect to re-plated hepatic endoderm (hepatocytes are not usually replatable). Additionally the polyurethane surface plays an instructive effect/role in maintaining hepatocyte identity and stable function for at least 15 days post-replating. The bio-active nature of the polyurethane surface, formed by polymerising PHNGAD, MDI and an extender, utilised in these studies may also be applicable to other eukaryotic, especially mammalian cell types and therefore provide generic and defined extra-cellular support.

In one embodiment of the invention the polymer is a polyurethane polymer formed from polymerising PHNGAD, MDI and an extender molecule.

PHNGAD is Poly[1,6-hexanediol/neopentyl glycol/di(ethylene glycol)-alt-adipic acid]diol.

MDI is 4,4′-Methylenebis(phenyl isocyanate) and the extender serves to increase physical parameters, such as elasticity, wettability and/or surface topography and/or biochemical properties such as the ability to absorb extracellular matrix proteins.

Suitable extender molecules include 1,4-butanediol (BD); 3-dimethylamino-1,2-propanediol (DMAPD); 3-diethylamino-1,2-propanediol (DEAPD); (BD), 2,2,3,3,4,4,5,5-octafluoro-1,6-hexanediol (OFHD), 1,3-propylene glycol (PG), 1,2-ethylene glycol (EG), 2-nitro-2-methyl-1,3-propanediol (NMPD), diethyl-bis-(hydroxymethyl)-malonate (DHM), 1,12 dedecanediol, cyclododecanediol, hydroquinone bis(2-hydroxylethyl)ether, 2,2,3,3-tetrafluoro-1,4-butanediol, 2,2,3,3-tetrafluoro-1,4-butanediol, 2-ethyl-1,3-hexanediol (EHD), N,N-diisopropanolanaline (DIPA), ethylenediamine, m-phenylene 4-diaminosulfonic acid (PDSA).

The present inventors have observed that polymers formed from all three components (PHNGAD, MDI and extender) are able to allow attachment of hepatocytes which display appropriate functional activity, whereas polymers which do not comprise an extender and are only formed from PHNGAD and MDI do not bind hepatocyte cells.

A preferred polymer for use in the present invention is identified herein as polymer 134 (see FIG. 5), although other related polymers (polymers 103, 104 and 247)—see Table 1, also display suitable properties in terms of adhesion and function of hepatocytes.

Hepatocytes as used herein may include hepatocyte cells which have been obtained directly from the liver, by way of for example a biopsy, of a subject. However, preferred hepatocytes are derived from embryonic stem cells or embryonic stem cell lines which have been differentiated into hepatocytes or hepatocyte like cells. Examples of such cells are described in [3]. Also hepatocyte like cells which have been obtained from reprogrammed adult cells [6] known in the art, may be employed (Takahashi & Yamanaka, (2006), Cell, 126, p663-676 and Takahashi et al, (2007), Cell, 131, p861-872).

In a further aspect there is provided use of a polymer as described herein for attachment of functional hepatocytes.

As used herein the terms “function” or “functional” refers to metabolic activity commonly associated with hepatocytes. Thus, the hepatocytes of the present invention desirably display more active endocrine and exocrine functions: the elevated production of human serum proteins—Fibronectin, Fibrinogen and Transthyretin and the expression of one or more cytochrome p450 enzymes, such as CYP3A4 and CYP1A2 are key examples. Moreover, such metabolic activity may be increased and/or be of longer duration than when the cells are attached to other substrates. Both MG and 134 show differing levels of hepatocyte maintenance 15 days post-replating, with 134 exhibiting ˜2 fold increase in CYP3A4, Fibronectin, Fibrinogen, Transthyretin and ˜6 fold increase in CYP1A2 activity.

The hepatocyte cells may be attached directly to the polymer of the present invention, with the polymer being formed into a suitable form. Alternatively the polymer may be physically or chemically coated using appropriate techniques such as spin coating, grafting or dip coating onto a suitable substrate. One of skill in the art will appreciate that spin coating is used to coat 2D- and 3D-substrates by spinning the substrate at certain rpm while a solution of the material used to coat the substrate is deposited on top of the surface. One of skill in the art will appreciate that dip coating comprises immersing a substrate into a solution of material used to coat the substrate at a certain velocity. One of skill in the art will appreciate that grafting consists of a chemical process between the substrate and the material to be used. The substrate provides a surface for polymeric coating. Examples of a suitable substrate include, but are not limited to, polymeric and ceramic materials, glasses, ceramics, natural fibres, synthetic fibres, silicones, metals, and composites thereof. In accordance with one embodiment of the present invention, the substrate may be fabricated of a polymeric material, such as polypropylene, polystyrene, polycarbonate, polyethylene, polysulfone, PVDF, Teflon, their composites, blends, or derivatives and the polyfibre core of a bio-artificial liver—a non-woven hydrophilic polyester matrix which is conducive to the immobilisation and high-density cultivation of hepatocytes.

The polymer or polymer coated substrate may take any suitable form and may be made into a porous or non-porous form. In accordance with another embodiment of the present invention, polymer or polymer coated substrate of the present invention may be in a form of threads, sheets, films, gels, membranes, beads, plates and like structures. In accordance with a further embodiment of the present invention, the polymer or polymer coated substrate may be fabricated in the form of a planar device having discrete isolated areas in the form of wells, troughs, pedestals, hydrophobic or hydrophilic patches, diecut adhesive reservoirs or laminated gasket diecuts that form wells, or other physical barriers to fluid flow. Examples of such a solid support include, but are not limited to, a microplate or the like.

In essence any suitable structure may be envisaged providing the hepatocyte cells are able to attach thereto. For example, in one embodiment the polymer may be coated onto wells formed in a microplate or printed in discrete locations on a substrate such that ordered assays of hepatocytes can be formed so as to allow testing of drugs and the like. It may also be appropriate to coat the substrate initially or in the areas to which the polymer is not bound, with a material which inhibits cell adhesion.

In another embodiment, the polymer or polymer coated substrate may take the form of a device designed to act as a bio-artificial liver or detoxifier which is designed to be used to metabolise agents which are added to it. Such a device may find application as a temporary device in subjects who have damaged livers. Alternatively, it may be used to identify metabolites of chemical agents, which may be of therapeutic use.

The present invention will now be further described by way of example and with reference to the Figures which show:

### FIGURE LEGENDS

FIG. 1: Screening a polymer library. (A) hESCs were differentiated to HE using an efficient differentiation protocol [3]. Abbreviations—bFGF—basic fibroblast growth factor; MEF-CM—mouse embryonic fibroblast conditioned medium; KO DMEM—knock out Dulbecco's Modified Eagle Medium; DMSO—Dimethyl sulfoxide; SR—serum replacement; L-15—Leibovitz's L-15; FCS—Fetal Calf Serum; HGF—Hepatocyte growth factor; OSM—oncostatin M; DAPI—4′,6-diamidino-2-phenylindole. (B) A. Phase contrast microscopy of cells replated on the polymer library following 8 days culture in hepatocyte culture medium (magnification ×10). B. Immunofluorescence for human albumin in hepatic endoderm (HE) replated on the polymer spots. C. Immunofluorescence for human albumin in HE maintained on matrigel. D. IgG isotype immunostaining control for human albumin staining. Photographs were taken using a Leica DMIRB, Scale Bar 100 μM. (C) Table of polymer hits compared to Matrigel control;

FIG. 2 shows Hepatocyte like cell functionality on matrigel vs defined polymer matrices.

hESCs were differentiated to hepatocyte like cells (HLCs) using an established method. At day 23 hESC-derived HLCs were incubated in 1 ml of hepatocyte culture medium for 24 hours. The following morning culture supernatants were harvested and serum protein production measured by ELISA and quoted as ng/mg of cellular protein. HLCs cultured on polymer 134 exhibited the greatest effect on hepatic function with a greater than 2 fold induction of fibrinogen (A), transthyretin (B) and fibronectin (C) when compared to the other polymers or matrigel extracellular matrices, (n=3);

FIG. 3 shows morphological and functional analysis of hepatocyte like cells plated on matrigel or identified polymers.

(A) Hepatic endoderm (HE) morphology was granular on all polymers screened, except for polymer 134 which exhibited a healthy morphology (see also FIG. 4A). (B) hESC-derived HE was incubated with hepatocyte culture media supplemented with 100 μM of CYP1A2 pGlo™ substrate as per manufacturers instructions. 4 hours post-treatment a 50 μl sample of the supernatant was removed and read on a luminometer (POLARstar optima). CYP1A2 activity was ˜6 fold greater on cells maintained on 134 than MG or the other polymers tested and activity is expressed as relative light units (R.L.U.)/mg protein, (n=3). (C) hESC-derived HE was incubated with hepatocyte culture media supplemented with 50 μM of CYP3A4 pGlo™ substrate as per manufacturers instructions. 5 hours post-treatment a 50 μl sample of the supernatant was removed and read on a luminometer (POLARstar optima). CYP3A4 activity was less than polymer 134, but was similar in HE maintained on the other polymers tested (see FIG. 4C). CYP3A4 activity is expressed as relative light units (R.L.U.)/mg protein, (n=3). (D) Cell lysates were Western blotted and probed for the human Pregnane X Receptor (hPXR), human albumin and beta-actin. hPXR and albumin production was greater in HLCs maintained on polymers 9G7 and 223 than HLCs maintained on polymers 2BG9, 212 and 3AA7;

FIG. 4 shows Hepatocyte morphology and function is preserved on polymer 134 vs Matrigel.

(A) hESC-derived HLCs morphology plated on matrigel (MG) or polyurethane 134 were compared. In general hepatocytes maintained on 134 looked healthier with a less grainy appearance. (B) Protein lysates were prepared from HE maintained on MG or 134. Extracts were Western blotted, blocked and probed for p-Akt, p-FAK, p-ERK, p15, p21, E-Cadherin, N-Cadherin, Albumin, hPXR and Cyp3A4. hESC-HE maintained on polymer 134, but not MG, displayed increased, Akt, FAK and ERK signalling; cell cycle inhibitors, p15 and p21, expression; adhesion molecule, E-Cadherin and N-Cadherin, expression and hPXR and Cyp3A4 expression. 2 house keeping genes, B-Actin and GAPDH, were employed as loading controls. Similar expression of B-Actin was observed in both MG and 134 samples, whereas greater expression of GAPDH was detected in MG protein samples. In addition HE maintained on polymer 134 exhibited the presence of a phosphorylated upper band consistent with drug inducible hPXR function. The levels of albumin remained similar on hepatocyte like cells maintained on polyurethane 134 and matrigel. (C) hESC-derived HLCs and primary human hepatocytes were incubated with hepatocyte culture media supplemented with 50 μM of CYP3A4 pGlo™ substrate as per manufacturers instructions. 5 hours post-treatment a 50 μl sample of the supernatant was removed and read on a luminometer (POLARstar optima). CYP3A4 activity was greater on cells maintained on 134 than MG and activity is expressed as relative light units (R.L.U.)/mg protein (* p<0.05 by the Student's t-test, (n=3)).

FIG. 5 shows the structure of polyurethane 134 and its constituent. For synthesis of this polyurethane, PHNGAD of 25% as polyol, MDI of 50% as diisocyanate and BD of 25% as a chain extender, were used.

FIG. 6 shows hESC derived HE either plated on an uncoated or coated polyfibre core (PFC) of the bio-artificial liver (BAL). At day 24 in culture the cells were fixed, stained and examined by electron microscopy. (a) is the uncoated PFC of the BAL. (b) represents uncoated PFC with cells attached, (c) represents the polymer 134 coated PFC and (d) represents polymer 134 coated PFC with cells attached. Scale bar in panel is 50 μM.

FIG. 7: (A) Prior to drug induction (days 17-21) hESC-derived HE was incubated in 1 ml of hepatocyte culture medium for 24 hours. The following morning culture supernatants were harvested and serum protein production measured by ELISA and quoted as ng/ml of culture supernatant (n=6) with a clear increase on polymer 134; and (B) hESC-derived HE was either plated in an uncoated (grey bars) or coated (black bars) polyfibre core (PFC) of the bio-artificial liver (BAL). The cultures were incubated in the presence (+) or absence (−) of a known CYP3A4 inducer, Phenobarbital (PB) (0.4 mM), for 48 hours prior to measurement of CYP3A4 activity. On day 24 hESC-derived HE was incubated with hepatocyte culture media supplemented with 50 μM of CYP3A4 pGlo™ substrate. 5 hours post-treatment CYP3A4 activity was measured on a luminometer (POLARstar optima), with clear induction of activity on polymer 134. Units of activity are expressed as relative light units (R.L.U.)/mg protein. * p<0.05 by the Student's t-test, (n=4).

FIG. 8: hESC-derived HE was either plated in an uncoated (Black bars) or coated bio-artificial liver matrix (Grey Bars). (A) The cultures were incubated in the presence ammonuium chloride for 4 hours prior to measurement of Urease activity. Urease activity is quoted at mM/mg cell protein/hr (n=12)* p<0.05; and (B) HE function on native and polymer coated bio-artificial liver matrix. Phenobarbital (SIGMA) drug induction was carried out from day 22 for 48 hours, changing media and Phenobarbital on a daily basis. Control cultures did not receive Phenobarbital, but had their media changed daily. The cultures were incubated in the presence or absence of a known CYP3A4 inducer, Phenobarbital (0.4 mM-5 mM), for 48 hours prior to measurement of CYP3A4 activity. On day 24 hESC-derived HE was incubated with hepatocyte culture media supplemented with 50 μM of CYP3A4 pGlo™ substrate (Promega—using non-lytic CYP450 activity estimation). 5 hours post-treatment CYP3A4 activity was measured on a luminometer (POLARstar optima). Units of activity are expressed as relative light units (R.L.U.)/mg protein (n=6).

FIG. 9: iPSC-derived HE CYP3A4 function on MG and polymer 134. iPSC-derived HE was cultured on matrigel and polymer 134. At day 24 it was assessed whether HE maintained on matrigel and polymer 134 displayed CYP3A4 function. Importantly, iPSC-derived HE re-plated on polymer 134 displayed significantly higher basal levels of CYP3A4 activity than those re-plated on matrigel. On day 24 hESC-derived HE was incubated with hepatocyte culture media supplemented with 50 μM of CYP3A4 pGlo™ substrate (Promega—using non-lytic CYP450 activity estimation). 5 hours post-treatment CYP3A4 activity was measured on a luminometer (POLARstar optima). Units of activity are expressed as relative light units (R.L.U.)/mg protein (n number is shown on the graph)

### MATERIALS AND METHODS

### Synthesis of PHNGAD (See Schemes 1 and 2 Below)

Diethylene glycol, 1,6-hexanediol, neopentyl glycol and adipic acid were purchased from Aldrich. Stannous octoate and titanium(IV) butoxide were of commercial grade (Aldrich), and used without further purification. 4,4′-methylenebis(phenylisocyanate) (MDI) was used as a diisocyanate and the chain extenders (3-dimethylamino-1,2-propanediol (DMAPD), 3-diethylamino-1,2-propanediol (DEAPD), 1,4-butanediol (BD) and 2,2,3,3,4,4,5,5-octafluoro-1,6-hexanediol (OFHD)) were used for polyurethane synthesis (Aldrich).

The synthesis of the PHNGAD polyol was performed using a melting technique of the monomers without any organic solvents. Initially, all monomers were subjected to heat treatment at 60° C. for 48 hours under vacuum to ensure the removal of water. The required amount of monomers, 1,6-hexanediol (0.22 mol), di(ethylene glycol) (0.22 mol), neopentyl glycol (0.22 mol) and adipic acid (0.55 mol) were charged into the reaction flask. The whole assembly was kept in an oven at 40° C. for 6 hours, to avoid any moisture absorption during charging the chemical into the flask. Following drying the required amount of catalyst either stannous octoate or titanium (IV) butoxide was injected through a needle, drop by drop, and the reaction mixture was heated to 180° C., stirred under N₂ atmosphere and water was collected through a condenser. The reaction was performed up to the desired time. The molecular weight distribution of the polyol can be controlled by varying the compositions of the monomers, catalyst, reaction time and temperature.

An alternative method for synthesis of the polyol is the solution technique, in which the monomers and other additives are dissolved in an organic solvent. In this case the reaction can be performed at much lower temperature than the melting one, and the solvents removed by evaporation.

### Synthesis of Polyurethanes

The synthesis of polyurethanes was performed by a two-step polymerisation method. The polyol of one equivalent was first reacted with two equivalents of diisocyante, and subsequently one equivalent of a chain extender added to the reaction solution to give copolymer product.

One or more catalysts may be used in the polyurethane synthesis. Particularly preferred catalysts are dibutyltin dilaurate, dimethyltin dicarboxylate, stannous octoate, iron(III) acetylacetonate. The preferred amount of catalysts are in the range of between 0 to 5% by weight.

Additionally, further additives may also be added such as antifoams or adhesive promoters during the reaction, which reduces the surface tension of a solution, thus inhibiting or modifying the formation of a foam.

Various solvents such as N,N′-dimethyl formamide (DMF), toluene, tetrahydrofuran (THF), chloroform, N-methyl-2-pyrrolidone (NMP), 1,2-dichloroethane, dioxane, dimethyl sulfoxide (DMSO), etc may be used. One or more solvents may be used to dissolve the starting materials in the reaction system, and the solvents are dry. Binary solvents may also be used in the synthesis of polyurethane.

The synthesis of polyurethane's may be performed at various temperatures, and the particular preferable temperature range is from 50° C. to 140° C. The reaction may be prolonged up to 96 hours, in an inert atmosphere, and preferably with N₂ or Argon purging.

Following the reaction, the polyurethane was collected by precipitation in which a poor solvent can be added drop wise into the reaction solution until the precipitation occurs. Finally, the polyurethanes were separated from the solution and analysed.

Various analytical techniques and methods were used to characterise these materials (such as gel permeation chromatography (GPC), NMR, FTIR spectroscope, differential scanning calorimeter (DSC), etc), to assure the molecular weight distribution and the functional groups of the polymers, and the melting and glass transition temperature, etc).

Various molecular weight distributions (Mw, Mn and D) (table 1) of polyurethanes may be achieved by varying the reaction conditions (such as reaction time, concentration of initiator, etc), the example of molecular weight range 5 kDa to 400 kDa preferable for this invention.

### Cell Culture

hESC culture was carried out as previously described [2,3]. hESCs were differentiated to hepatocyte like cells using activin and wnt3a as published [3]. At day 9 in the differentiation process the cells were removed from their substrate using a 5 minute 37° C. incubation with Trypsin/EDTA (Invitrogen). Following this hepatocyte like cells were seeded onto the polymer array, polymer coated coverslips or matrigel coated plasticware. The iPS cell line 33D-6 was cultured, propagated and differentiated to hepatic endoderm as previously described [6]. At day 9 in the differentiation protocol the cells were removed from their substrate by a 5 minute incubation with Trypsin/EDTA (Invitrogen). Following this hepatocyte like cells (HLCs) were plated onto polymer 134 or MG, and cultured in L-15 maturation medium as [6].

### Immunostaining

Immunostaining was carried out as previously described [3].

### ELISA

ELISAs were carried out as previously described [3].

p450 Assay

CYP3A4 and CYP1A2 activity were assessed using the pGlo kit from Promega and carried out as per manufacturers instructions for non-lytic CYP450 activity estimation, (http://www.promega.com/tbs/tb325/tb325.pdf). CYP Activities are expressed as relative light units (RLU) per milligram of protein.

### Western Blotting

Western blotting was carried out as previously described [4]. Primary antibodies to the proteins are shown in the table below:

| Antigen | Company | Dilution |
|---|---|---|
| p-FAK | Epitomics | 1:5000 |
| p-Akt | Cell Signaling | 1:4000 |
| p-Erk | Cell Signaling | 1:4000 |
| P15 | Cell Signaling | 1:1000 |
| P21 | Santa Cruz | 1:500 |
| E-Cad | DAKO | 1:1000 |
| N-Cad | DAKO | 1:1000 |
| h-PXR | ABCAM | 1:250 |
| CYP3A4 | Dundee University | 1:250 |
| Albumin | SIGMA | 1:1000 |
| B-Actin | Santa Cruz | 1:2000 |
| GAPDH | Santa Cruz | 1:2000 |
| Secondary | DAKO Anti Mouse | 1:2000 |
| Secondary | DAKO Anti Sheep | 1:2000 |
| Secondary | DAKO Anti Rabbit | 1:2000 |

### Polymer Screening

Coverslips were coated with the six polymers as previously reported [7].

### Synthesis of PHNGAD and PU-134

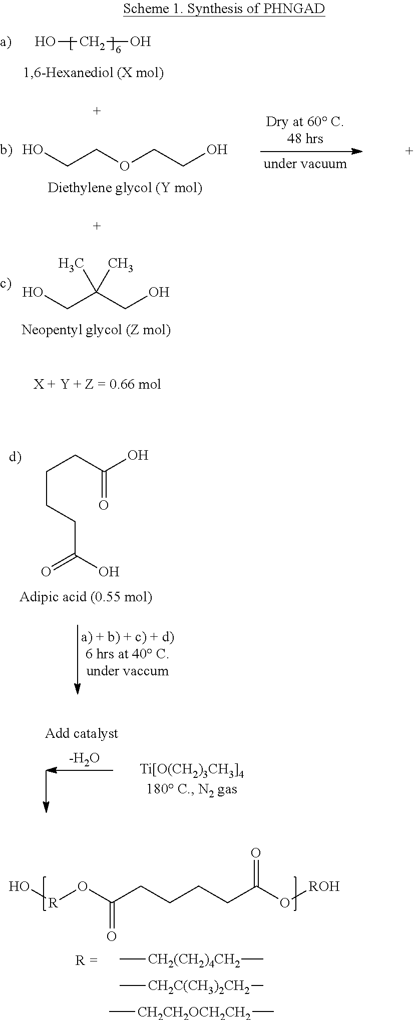

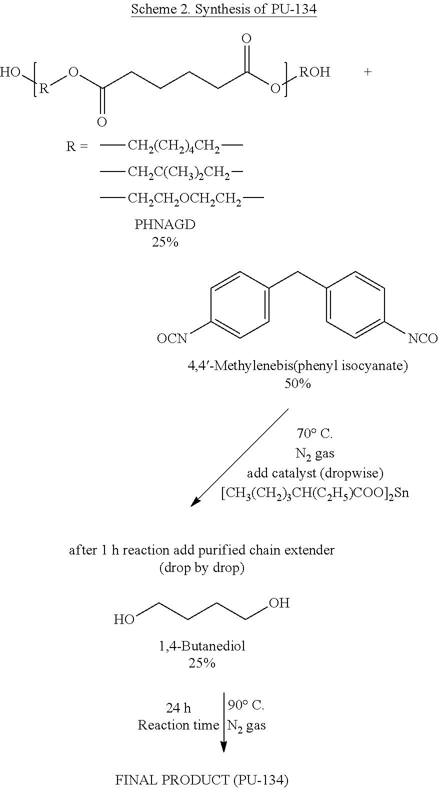

### EXAMPLE 1

### Polymer Library Screening and Characterisation

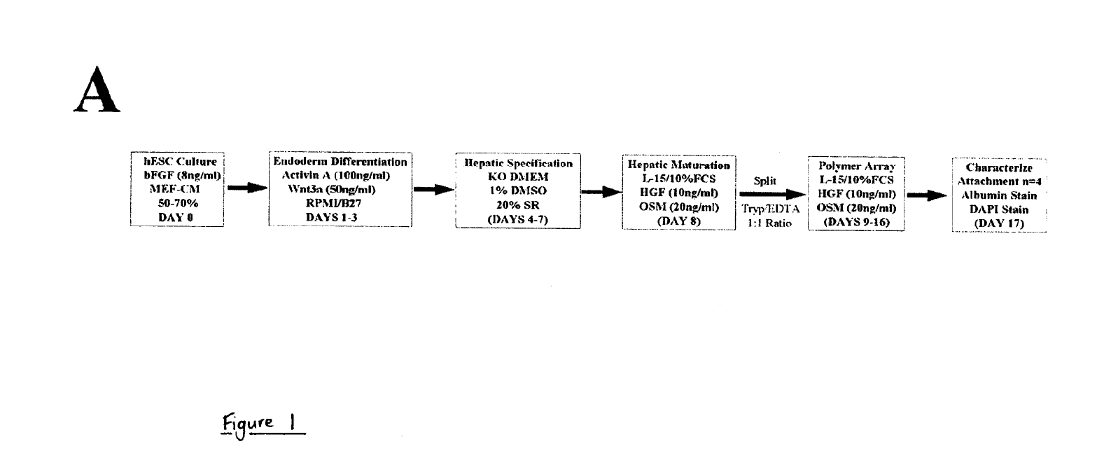

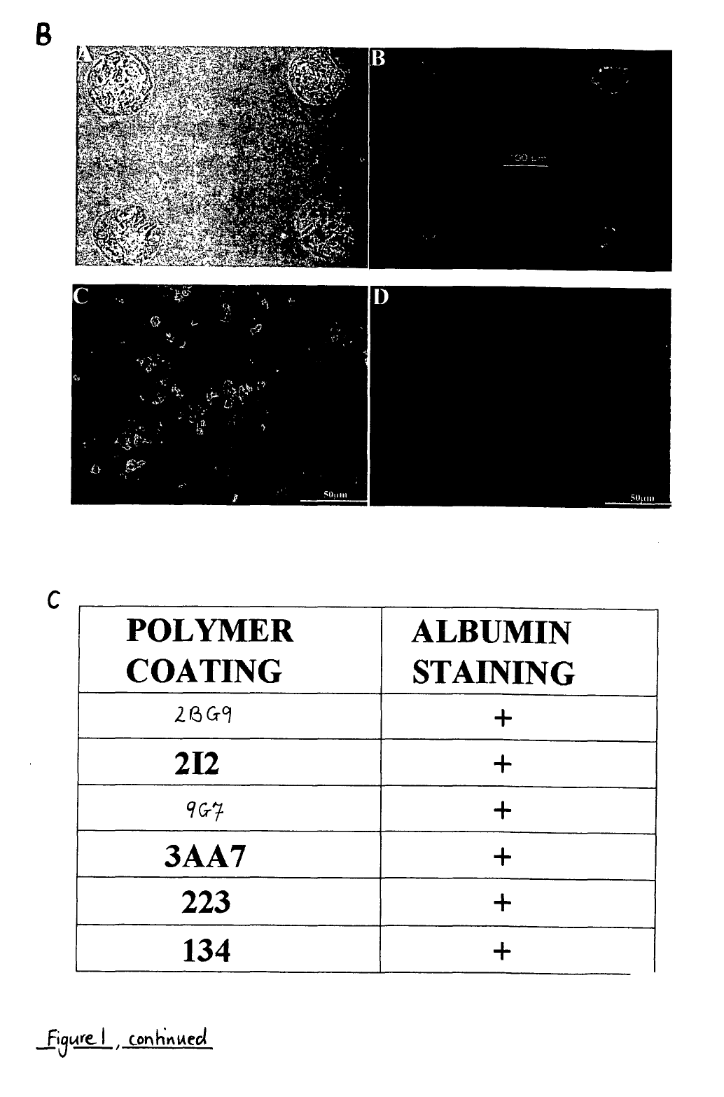

Polymer microarrays were fabricated by contact printing 380 generic polyurethane and polyacrylate polymers onto an agarose coated glass microscope slide [7, 8]. Once printed, the slides were dried overnight and sterilised by UV irradiation prior to cell plating. We screened this polymer library for stem cell derived hepatic endoderm (HE) attachment, stabilisation and promotion of function. Direct differentiation of human embryonic stem cells (hESCs) to HE was initiated using a recently developed highly efficient tissue culture model (FIG. 1A). Upon adopting a hepatic fate (Day 9), HE was detached from their biological extracellular matrix and replated onto the polymer array/library. Following replating onto the array, hESC-derived HE was cultured for a further 8 days in conditions that support hepatic identity and differentiation in vitro. At this point cell attachment was recorded using phase microscopy (FIG. 1B, A); hepatic phenotype and function was assessed by albumin production (FIG. 1B. B); and compared to current “gold standard” conditions (culture on Matrigel, FIG. 1B, C) and an IgG isotype control (FIG. 1B, D). Primary screening identified polymers that supported HLC attachment and identity (FIG. 1C).

### EXAMPLE 2

### Evaluation of Selected Polymers

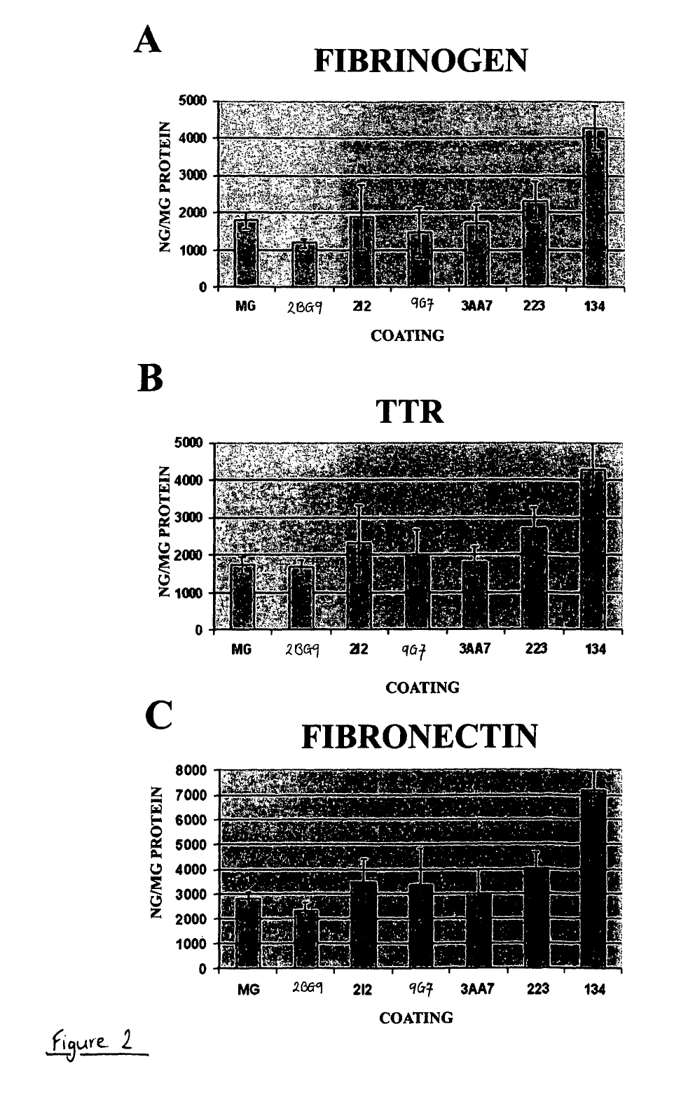

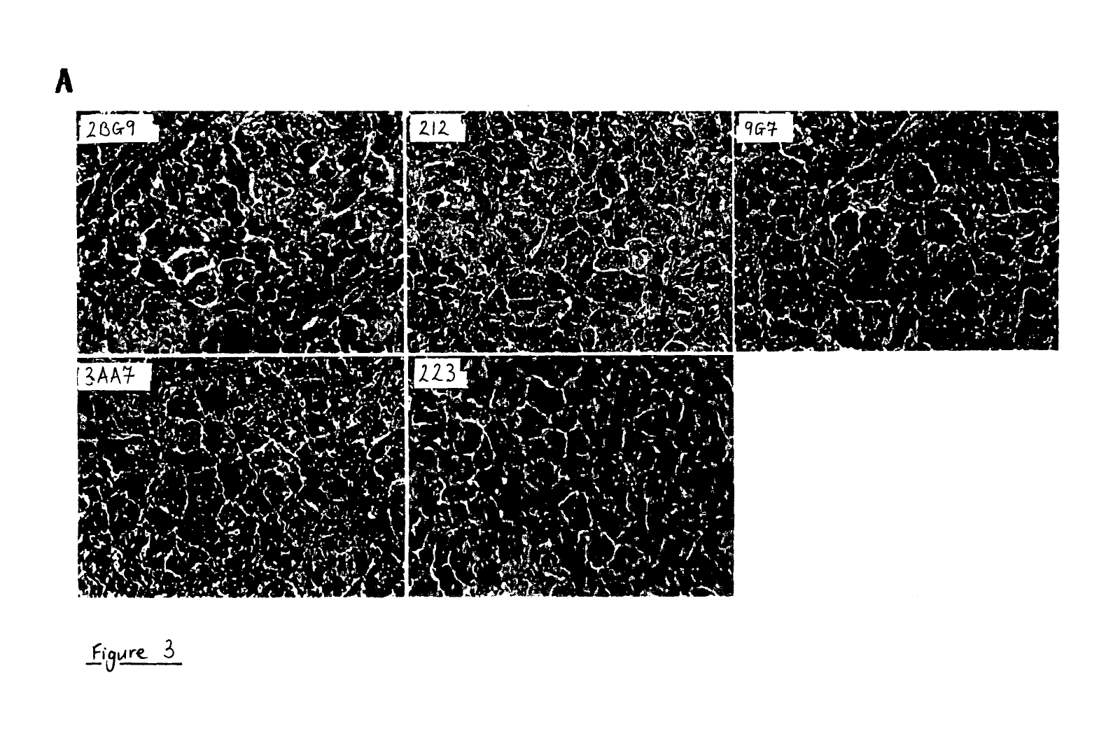

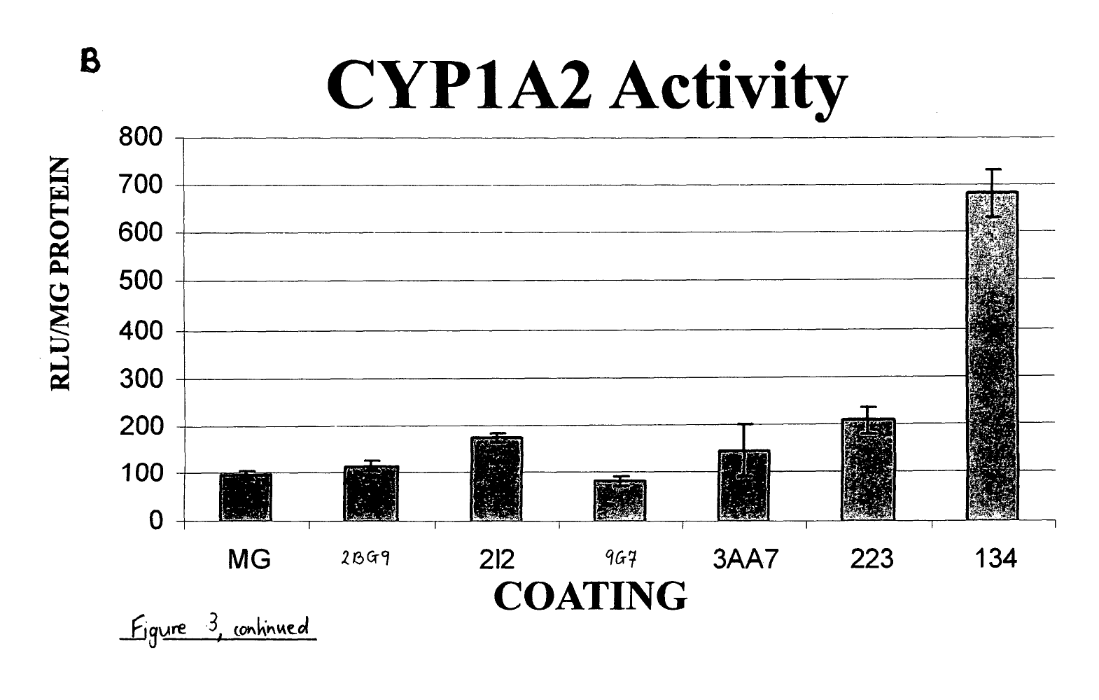

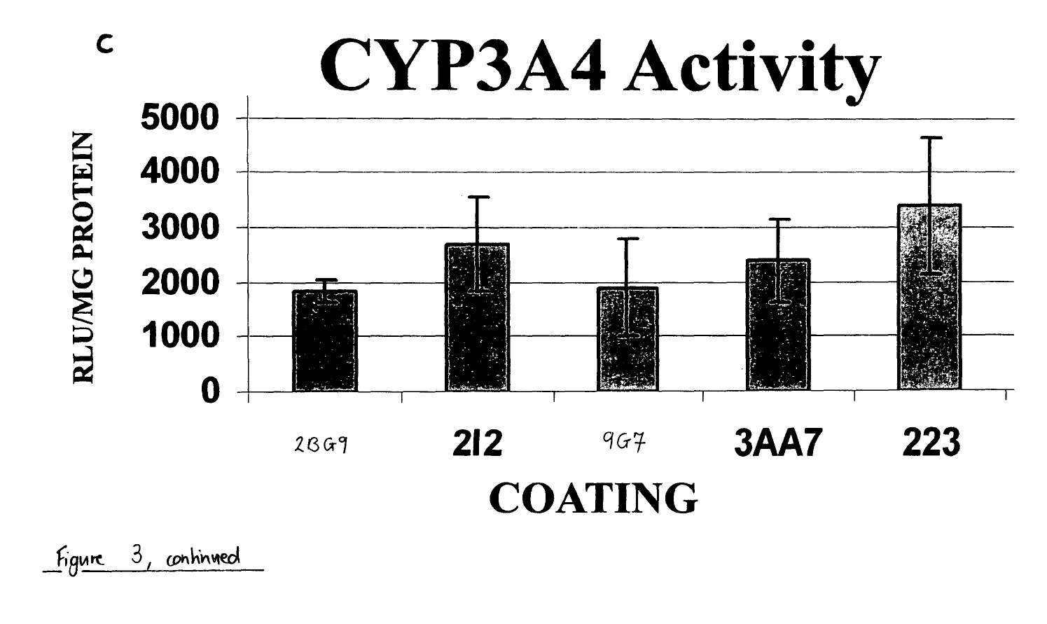

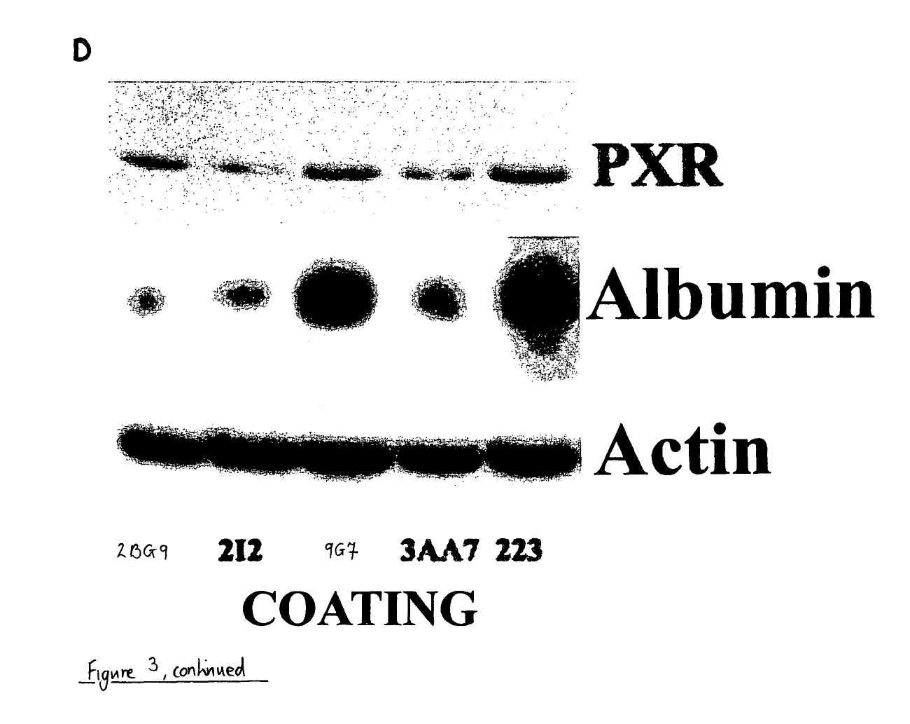

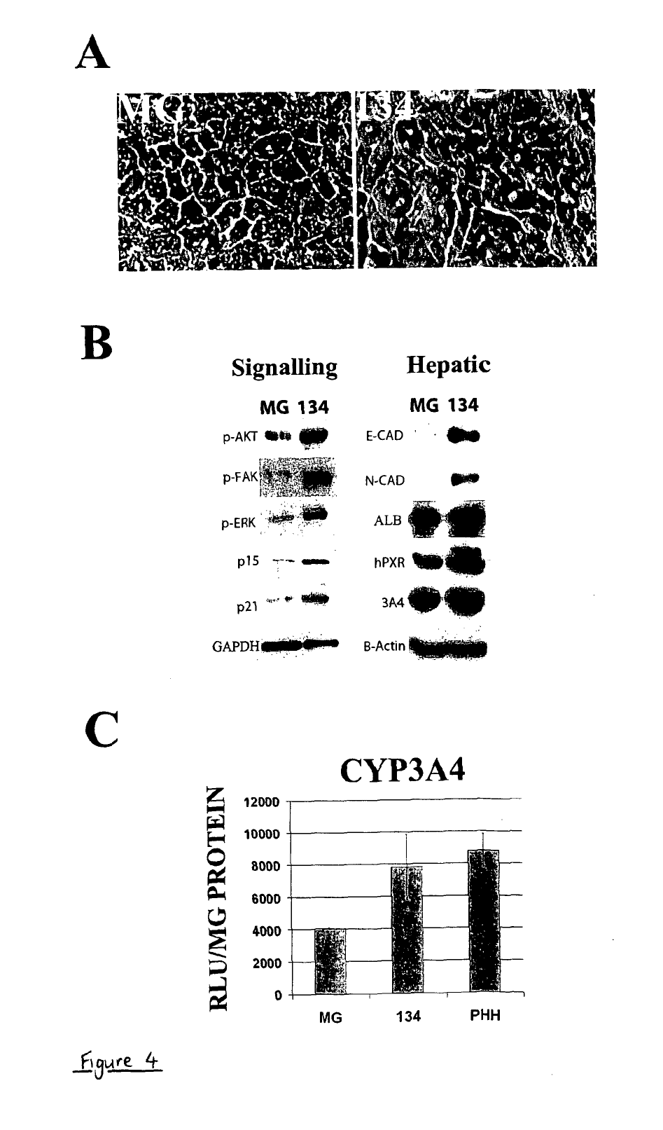

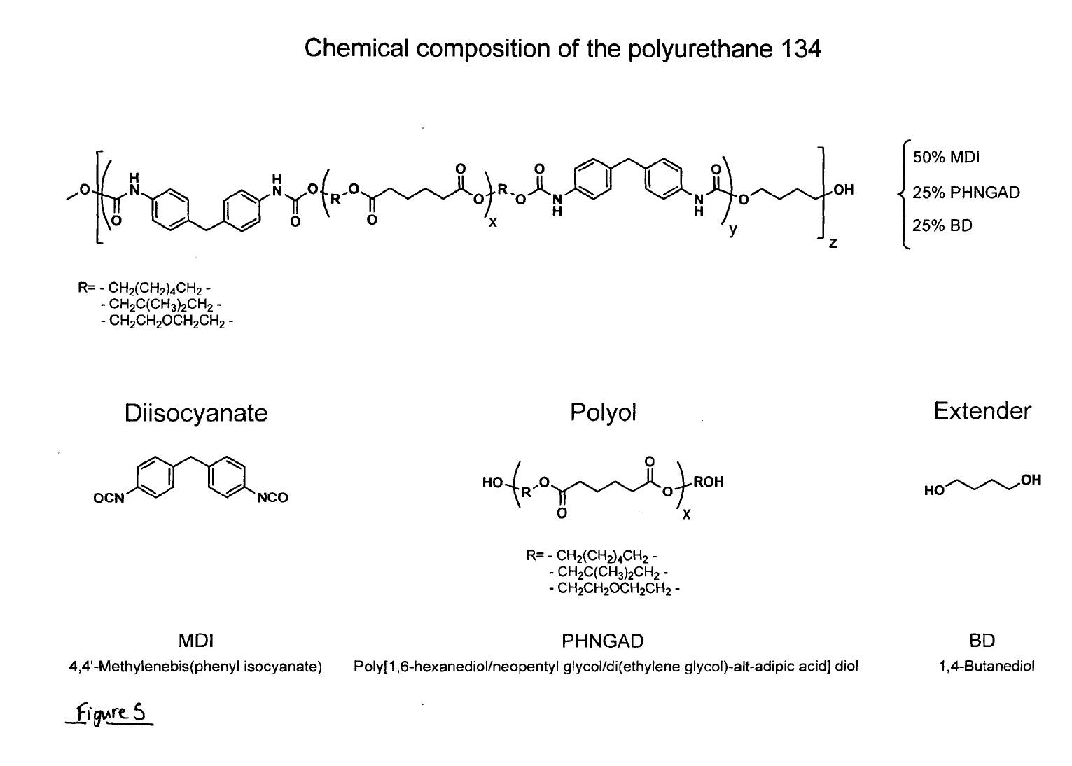

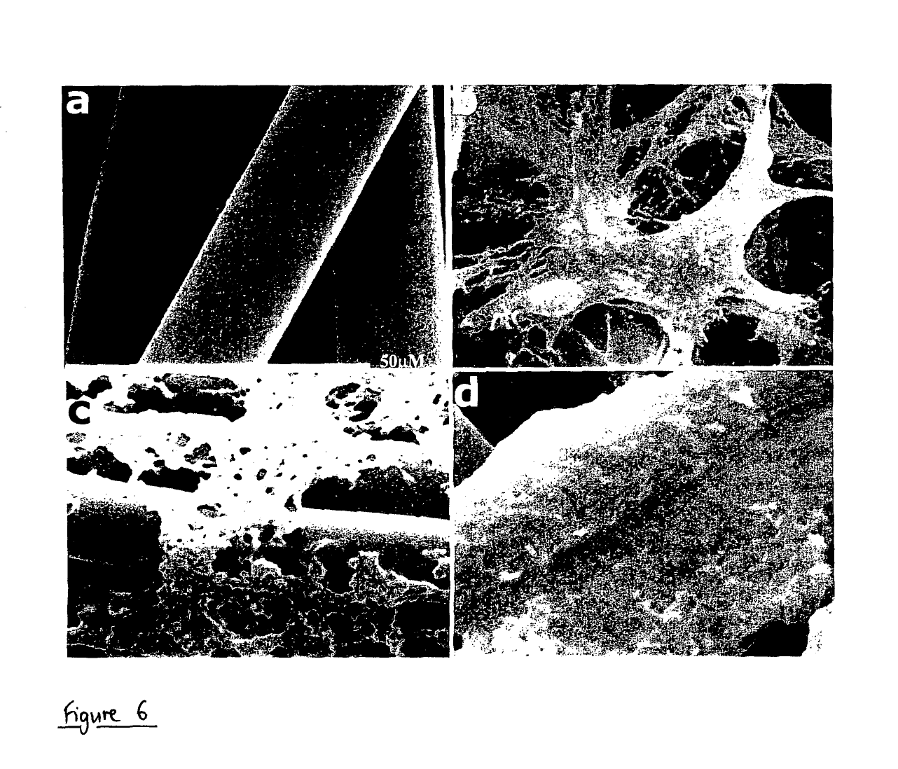

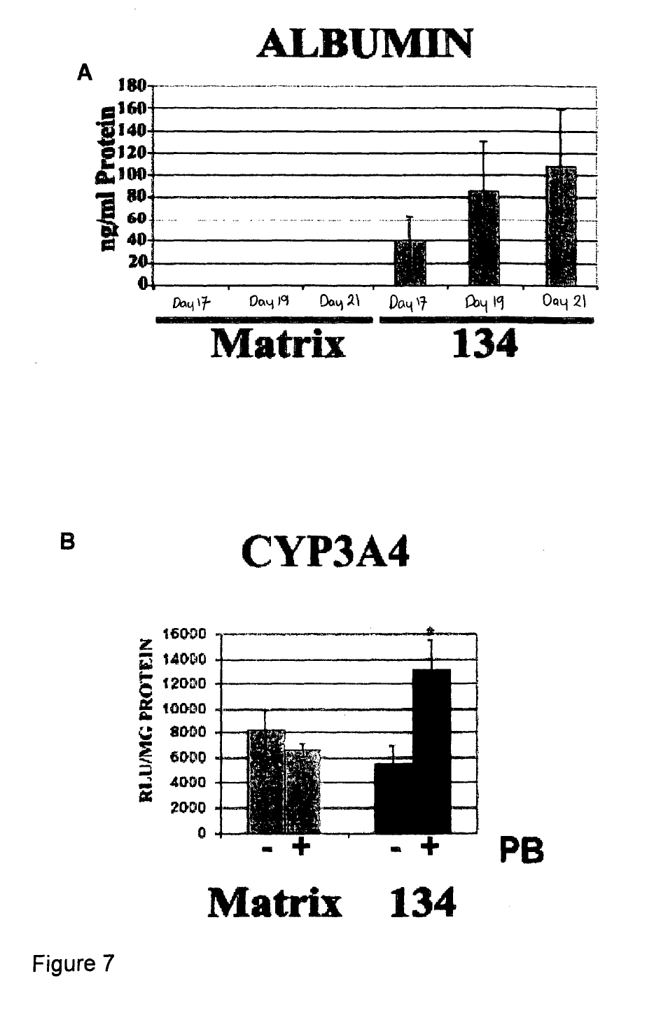

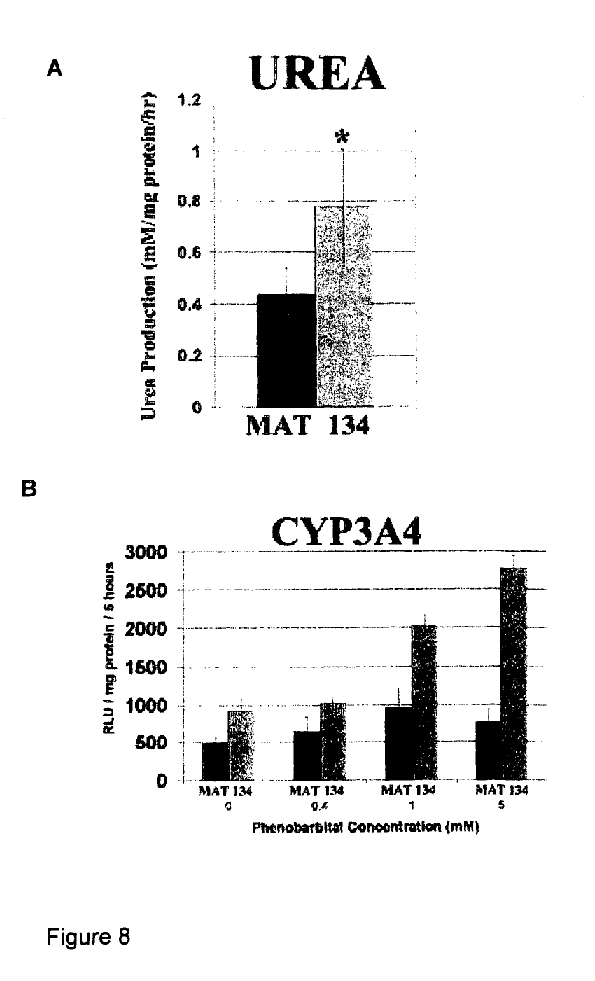

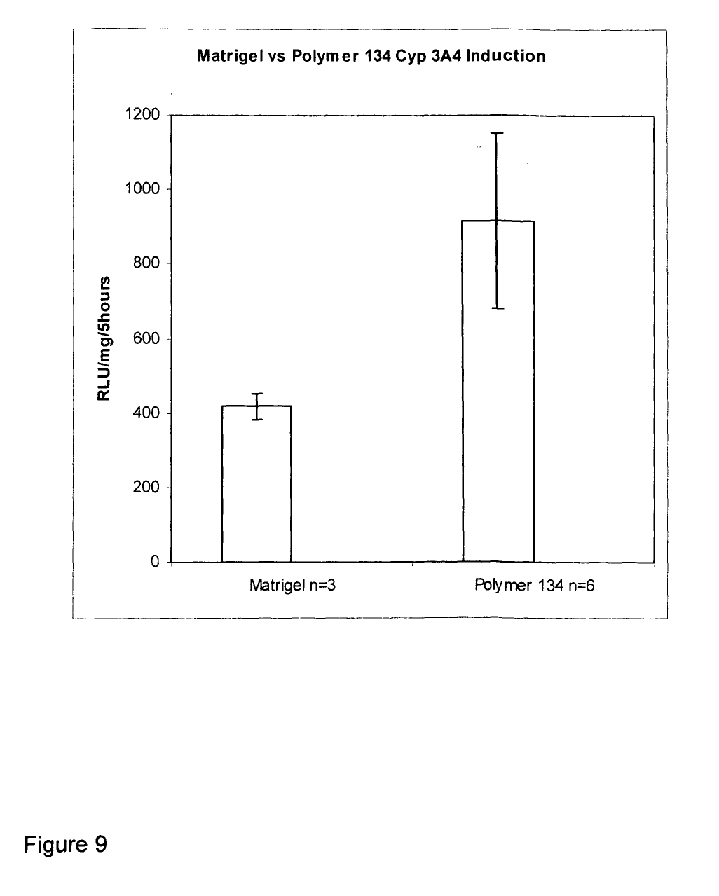

Human-ESCs were differentiated and replated as detailed. Cell function was assessed at 15 days post re-plating and defined by expression of a panel of hepatocyte specific genes and export of essential serum proteins. Using this strategy, polymer 134 [7] was identified as the most effective cellular support associated with enhanced expression of Fibrinogen [9] (FIG. 2A), transthyretin (TTR) [10] (FIG. 2B) and soluble fibronectin [11] (FIG. 2C). We also observed a significant change in HLC morphology between the two ECMs (day 15). HLCs passaged and maintained on matrigel or polymers 2BG9, 212, 9G7, 3AA7 and 223 (FIG. 3A and FIG. 4A) were granular. In contrast, HLCs replated on 134 maintained a clear hepatic morphology (FIG. 4A). We then analysed key cytochrome p450 (CYP) activities on the different extra-cellular matrices. CYP1A2 activity was increased ˜6 fold on polymer 134 as compared to standard matrigel conditions or the other polymers assessed (FIG. 3B). We also investigated CYP3A4 function as it plays a fundamental role in a number of exocrine pathways and is involved in the metabolism of approximately 50% of prescribed drugs. Many drugs are also known to have CYP3A4 inhibiting activities making this an attractive target molecule in the drug discovery process [12]. HLCs maintained on polymer 134 displayed ˜2 fold increase in CYP3A4 p450 function over cells maintained on matrigel or the other polymers tested (FIG. 4C, FIG. 3C). The human pregnane receptor (hPXR) is a key regulator of CYP3A gene expression [13]. hPXR is sequestered in the cytoplasm [14] and upon ligand binding dissociates. During hPXR's nuclear translocation it is phosphorylated in a protein kinase A dependent manner [12]. Upon entry to the nucleus hPXR dimerizes with the retinoic acid receptor and regulates CYP3A expression via core elements contained within the CYP3A gene promoter [15]. In agreement with previous findings we demonstrate that HLCs maintained on 134 displayed increased levels of hPXR expression and phosphorylation co-inciding with peak levels of CYP3A4 expression and activity (FIG. 3C, FIG. 4C. We also analysed albumin expression in these experiments and demonstrated that gene expression of albumin was similar between HLCs maintained on matrigel and polymer 134. However albumin and hPXRexpression did vary considerably on the other polymers tested (FIG. 3D).

Following the observations of polymer 134 and its potential utility, further related polymers (prepared in accordance with earlier papers 7, 8) were studied and others identified (see Table 1) which also allow attachment and maintained function of hepatocytes.

### EXAMPLE 3

### Extensive Characterisation of hESC-Derived HE on Polymer 134 and Matrigel

Our further studies focussed on HE morphology, signalling, gene expression and drug metabolism on two extracellular matrices, Matrigel and polymer 134. Matrigel was used as our control as it has previously been shown to improve hepatocyte performance in vitro and is currently considered the “gold standard”. We observed a significant change in HE morphology (day 24), thus HE passaged and maintained on matrigel or polymers 2BG9, 212, 9G7, 3AA7 and 223 (FIG. 3A and FIG. 4A) were granular. In contrast, HE replated on 134 maintained a clear hepatic morphology (FIG. 4A). In line with changes in cellular morphology we also observed changes in general cell signalling and hepatic gene expression. hESC-HE maintained on polymer 134 displayed increased FAK, Akt and ERK signalling, consistent with the cells becoming firmly attached to their substrate and not under-going apoptosis. This was not observed in HE maintained on Matrigel. Although HE plated on polymer 134 displayed elevated levels of the active mitotic factor ERK, HE also expressed high levels of cell cycle inhibitors p15 and p21. Taken together these results are consistent with metabolically active hepato-cellular populations locked in a quiescent/functional state (FIG. 4B). In addition to changes in cell signalling and cell cycle we also observed changes in hepatocyte gene expression. The expression of both N-Cadherin and E-Cadherin play important roles in human hepatocyte biology. We observed enhanced expression of both of these molecules in hESC-HE maintained on 134 compared with MG. In addition to a more epithelial phenotype on polymer 134, we also observed that replated HE also displayed increased expression of hPXR and phospho-PXR on polymer 134. The increased levels of hPXR were consistent with increased levels of cytochrome p450 (CYP) 3A4 detected by Western blotting (FIG. 4B). In order to assess the role that polymer was playing in CYP3A4 metabolic activity we analysed key activities on the different extracellular matrices and compared to primary human hepatocytes. HE maintained on polymer 134 displayed similar CYP3A4 p450 function to primary human hepatocytes (PHH) maintained on matrigel and ˜2 fold increase in CYP3A4 p450 function over hESC-derived HE maintained on matrigel or the other polymers tested (FIG. 4C and FIG. 3C). In addition to CYP3A4 we also tested CYP1A2 activity which was induced ˜6 fold on polymer 134 as compared to standard matrigel conditions or the other polymers assessed (FIG. 3B). We also analysed albumin expression in these experiments and demonstrated that expression of albumin was similar between HE maintained on matrigel and polymer 134 (FIG. 4B);

### EXAMPLE 4

### Attachment of hESC-Derived He onto Native and Polymer 134 Coated Bioartifical Liver Matrix

We employed the polyfibre core (PFC), the cell matrix, used in a bio-artificial liver (BAL) device. The PFC was used in its native form or coated with polymer 134. Upon adopting a hepatic fate (Day 9), HE was detached from the biological extracellular matrix and replated onto native or polymer coated PFC and cultured for a further 15 days (day 24) in conditions that support hepatic identity. At day 24 we fixed HE attached to the uncoated (FIG. 6Aa) and polymer coated BAL matrix (FIG. 6Ac) and examined cell structure by electron microscopy. hESC-derived HE maintained on uncoated PFC demonstrated cell attachment and cell processes resembling stress fibres (FIG. 6Ab) whereas HE maintained on polymer 134 coated PFC exhibited a smooth tissue like appearance (FIG. 6Ad) which may limit the effects of fluid shear stress on HE in the BAL.

### EXAMPLE 5

### hESC-Derived HE Function on Native and Polymer 134 Coated Bio-Artificial Liver Matrix

These data exemplify the value of polymer 134 and hESC-derived HE in a BAL setting. In addition to p450 drug inducibility polymer 134 also promoted human albumin production measured prior to drug induction (days 17 to 21) (FIG. 7A). hESC-derived HE was cultured on the different BAL PFC substrates for 13 days, before we assessed HE drug inducibility (days 22-24). hESC-derived HE was induced with 0.4 mM Phenobarbital or maintained in control media for 48 hours, changing media daily. At day 24 we assessed whether the HE maintained on both the PFC support matrices displayed CYP3A4 drug induction. The uncoated PFC supported hepatocyte attachment and function, however the cellular niche did not support. Phenobarbital drug induction of CYP3A4 (FIG. 7B, Matrix PB+). In contrast hESC-HE replated onto polymer 134 coated supported both HE attachment and phenobarbital inducible CYP3A4 drug metabolism (FIG. 7B, 134 PB+).

### EXAMPLE 6

### hESC-Derived HE Function on Native and Polymer 134 Coated Bio-Artificial Liver Matrix

These data exemplify the value of polymer 134 and hESC-derived HE in a BAL setting. In addition to p450 drug inducibility polymer 134 also promoted human urease activity (day 24) (FIG. 8A). hESC-derived HE was cultured on the different BAL PFC matrices (+/−Polymer 134) substrates for 13 days, before we assessed HE drug inducibility (days 22-24). hESC-derived HE was induced with a range of Phenobarbital concentrations (0.4 mM-5 mM) or maintained in control media for 48 hours, changing media daily. At day 24 we assessed whether the HE maintained on both the PFC support matrices displayed CYP3A4 drug induction. At day 24 the native matrix supported hepatocyte attachment and function, with poor drug induction of CYP3A4 (FIG. 8B, Black Bars). In contrast, hESC-HE replated on polymer 134 coated matrix exhibited dose dependent phenobarbital inducible CYP3A4 drug metabolism (FIG. 8B, Grey Bars).

### EXAMPLE 7

### iPSC-Derived HE CYP3A4 Functionality on Native Substrate and Polymer 134 Coated Coverslips

A polyurethane matrix (polymer 134) plays an important role in hepatocyte functionality by facilitating the culture of highly functional iPSC-derived hepatic endoderm (HE). FIG. 9 shows that iPSCs can be differentiated to HE and replated on polymer 134 which promoted HE viability and functionality. In line with hESC derived HE, iPSC derived HE cultured on polymer 134 demonstrated higher basal levels of CYP3A4 activity than identical cells grown on matrigel.

In conclusion, screening allowed the identification of a new class of polymer matrix that promotes long-term hepatocellular differentiated function before and after passaging. These attributes bypass current limitations associated with adult human hepatocytes, and will play important roles in developing in vitro models of drug toxicology and may help to reduce drug attrition rates. Additionally our in vitro derived cells provide a resource for the construction of extra-corporeal devices and facilitate novel studies of human liver development and disease.

### REFERENCES

[1]Pharmaceutical Research and Manufacturers of America (PhRMA). Pharmaceutical Industry Profile 2005.
[2] Hay D C et al, 2008 Stem Cells. 2008 26(4): 894-902.
[3] Hay D C et al, 2008 Proc Natl Acad Sci USA. 2008 105(34): 12301-6.
[4] Hay D C et al, Cloning Stem Cells. 2007 (9): 51-62.
[5] Fletcher J et al, Cloning and Stem Cells. 2008 (10): 331-340.
[6] Sullivan et al, Hepatology. 2010 51(1):329-35
[7] Pernagallo S et al, Lab Chip. 2009 9: 397-403
[8] Uniciti-Broceta et al, J Comb Chem 2008: 10, 24-27.
[9] Tennent G A et al, Blood. 2007 109 (5):1971-4.
[10] Ingenbleek Y et al, Annu Rev Nutr. 1994 (14): 495-533.
[11] Pankov R et al, J Cell Sci. 2002 115 (Pt 20): 3861-3.
[12] Keshava C et a,l Am J Epidemiol. 2004 160 (9): 825-41.
[13] Ding X et al, J Pharmacol Exp Ther. 2005 312 (2): 849-56.
[14] Squires E J et al, J Biol. Chem. 2004 279 (47): 49307-14.
[15] Orans J et al, Mol Endocrinol. 2005 (12): 2891-900.

TABLE 1

| | Polymer structure | | | | | | Polymer property | | | | | |
|---|---|---|---|---|---|---|---|---|---|---|---|---|
| | | | | ratio (mol) | | | Molecular weight | | | Microarray screening | | |
| PU- | Diol | DIS | Extender | mon (1) | mon (2) | x | Mw | Mn | D | Screened | Adhesion | Hepatocytic Morpholgy |
| 103 | PHNGAD | MDI | DMAPD | 0.25 | 0.52 | 0.23 | 57000 | 22000 | 2.6 | Y | Y | Y |
| 104 | PHNGAD | MDI | DEAPD | 0.25 | 0.52 | 0.23 | 38000 | 17000 | 2.1 | Y | Y | Y |
| 134 | PHNGAD | MDI | BD | 0.25 | 0.52 | 0.23 | 72000 | 28000 | 2.6 | Y | Y | Y |
| 247 | PHNGAD | MDI | OFHD | 0.25 | 0.52 | 0.23 | 46000 | 22000 | 2.1 | Y | Y | Y |
| 248 | PHNGAD | MDI | none | 48.5 | 51.5 | 0 | 65000 | 26000 | 2.5 | Y | N | |

1-13. (canceled)

14. A polyurethane polymer comprising hepatocyte cells bound or adhered thereto.

15. The polyurethane polymer of claim 14, wherein the polymer is formed by polymerising Poly[1,6-hexanediol/neopentyl glycol/di(ethylene glycol)-alt-adipic acid]diol (PHNGAD); 4,4′-Methylenebis(phenyl isocyanate:MDI) and an extender.

16. The polyurethane polymer of claim 15, wherein the extender molecule is selected from the group consisting of 1,4-butanediol (BD); 3-dimethylamino-1,2-propanediol (DMAPD); 3-diethylamino-1,2-propanediol (DEAPD); (BD); 2,2,3,3,4,4,5,5-octafluoro-1,6-hexanediol (OFHD); 1,3-propylene glycol (PG); 1,2-ethylene glycol (EG); 2-nitro-2-methyl-1,3-propanediol (NMPD); diethyl-bis-(hydroxymethyl)-malonate (DHM); 1,12 dedecanediol; cyclododecanediol, hydroquinone bis(2-hydroxylethyl)ether; 2,2,3,3-tetrafluoro-1,4-butanediol; 2,2,3,3-tetrafluoro-1,4-butanediol; 2-ethyl-1,3-hexanediol (EHD); N,N-diisopropanolanaline (DIPA); ethylenediamine and m-phenylene 4-diaminosulfonic acid (PDSA).

17. The polyurethane polymer of claim 14, wherein the polymer is selected from the group consisting of polymer 103; 104; 134 and 247 shown in Table 1.

18. The polyurethane polymer of claim 14, wherein the hepatocyte cells are functional.

19. A substrate for culturing cells, coated with a polyurethane polymer.

20. The substrate of claim 19, wherein the substrate comprises a material selected from the group consisting of polypropylene; polystyrene; polycarbonate; polyethylene; polysulfone; PVDF; Teflon; and their composites, blends, or derivatives.

21. A bio-artificial liver or detoxifier comprising a polyurethane polymer.

22. The bio-artificial liver or detoxifier of claim 21, wherein the polyurethane polymer is formed from polymerising Poly[1,6-hexanediol/neopentyl glycol/di(ethylene glycol)-alt-adipic acid]diol (PHNGAD); 4,4′-Methylenebis(phenyl isocyanate:MDI) and an extender.

23. The bio-artificial liver or detoxifier of claim 22, wherein the extender molecule is selected from the group consisting of 1,4-butanediol (BD); 3-dimethylamino-1,2-propanediol (DMAPD); 3-diethylamino-1,2-propanediol (DEAPD); (BD); 2,2,3,3,4,4,5,5-octafluoro-1,6-hexanediol (OFHD); 1,3-propylene glycol (PG); 1,2-ethylene glycol (EG); 2-nitro-2-methyl-1,3-propanediol (NMPD); diethyl-bis-(hydroxymethyl)-malonate (DHM); 1,12 dedecanediol; cyclododecanediol, hydroquinone bis(2-hydroxylethyl)ether; 2,2,3,3-tetrafluoro-1,4-butanediol; 2,2,3,3-tetrafluoro-1,4-butanediol; 2-ethyl-1,3-hexanediol (EHD); N,N-diisopropanolanaline (DIPA); ethylenediamine and m-phenylene 4-diaminosulfonic acid (PDSA).

24. The bio-artificial liver or detoxifier of claim 21, wherein the polymer is selected from the group consisting of polymer 103; 104; 134; and 247 shown in Table 1.

25. The bio-artificial liver or detoxifier of claim 21, further comprising hepatocyte cells obtained from the liver, from embryonic stem cells, reprogrammed cells, from embryonic stem cell lines and/or hepatocyte like cells.

26. The polyurethane polymer of claim 14, wherein the hepatocyte is a hepatocyte cell obtained from the liver, from embryonic stem cells, from embryonic stem cell lines, reprogrammed cells which have been differentiated into hepatocytes and/or hepatocyte like cells.

27. The polyurethane polymer of claim 14, wherein the hepatocyte cells are attached directly to the polymer.

28. The polyurethane polymer of claim 14, wherein the polyurethane polymer is physically or chemically coated onto a suitable substrate.

29. The polyurethane polymer of claim 28 wherein the suitable substrate is selected from the group consisting of polymeric and ceramic materials; glasses; ceramics; natural fibres; synthetic fibres; silicones; metals; and composites thereof.

30. The polyurethane polymer of claim 28, wherein the suitable substrate is fabricated of a material, selected from the group consisting of polypropylene; polystyrene; polycarbonate; polyethylene; polysulfone; PVDF; Teflon; and their composites, blends, or derivatives.

31. The polyurethane polymer of claim 14, wherein the polyurethane polymer takes the form of a thread, sheet, film, gel membrane, bead or plate structure.

32. The polyurethane polymer of claim 14, wherein the polyurethane polymer forms a planar device having discrete isolated areas in the form of wells, troughs, pedestals, hydrophobic/hydrophilic patches or direct adhesive reservoirs/laminated gasket die cuts that form wells.

33. The polyurethane polymer of claim 28, wherein the suitable substrate is the polyfibre core of a bio-artificial liver.
